(19) United States
(12) Patent Application Publication
(10) Pub. No.: US 2007/0040995 A1
(43) Pub. Date: Feb. 22, 2007

(54) Synchronization of an image producing element and a light color modulator

(76) Inventor: Kyrre Tangen, Fredrikstad (NO)

Correspondence Address: ALLEMAN HALL MCCOY RUSSELL & TUTTLE LLP, 806 SW BROADWAY, SUITE 600, PORTLAND, OR, 97205-3335, US

(21) Appl. No.: 11/454,484

(22) Filed: Jun. 16, 2006

Related U.S. Application Data
(60) Provisional application No. 60/691,686, filed on Jun. 17, 2005.

(51) Int. Cl. A61B 8/00; G03B 21/14

(52) U.S. Cl. 353/84

(57) ABSTRACT

A display device is disclosed, wherein the display device includes a light source, an image producing element, a light color modulator disposed optically between the light source and the image producing element, a controller configured to synchronize the image producing element and the light color modulator, and a color transition detection system configured to provide a digitally enhanced representation of a light color transition to the controller.

### CROSS-REFERENCES TO RELATED APPLICATIONS

This application claims the benefit of U.S. Patent Provisional Application Ser. No. 60/691,686 filed Jun. 17, 2005 and entitled SYSTEM AND METHOD FOR SYNCHRONIZING A COLOR WHEEL AND A DIGITAL MICROMIRROR DEVICE, which is incorporated herein by reference in its entirety for all purposes.

### TECHNICAL FIELD

The present disclosure relates to the synchronization of an image producing element and a color modulator in a display device.

### BACKGROUND

In a display device such as a front or rear projection device, a color image may be produced by modulating white light from a light source as a function of time via a color modulator such as a color wheel. The modulated light may then be used to sequentially display red, green and blue images via a suitable image producing device, such as a digital micromirror device or a liquid crystal display, at a sufficiently high rate that the resulting sequence of separate red, green and blue images appears to be a single color image. Close synchronization of the modulator and image producing element may allow accurate image reproduction to be achieved. However, where the color modulator and the image producing element are not accurately synchronized, errors in the appearance and/or color of the image may result.

### SUMMARY

In one embodiment, a display device is provided, wherein the display device includes a light source, an image producing element, a light color modulator disposed optically between the light source and the image producing element, a controller configured to synchronize the image producing element and the light color modulator, and a color transition detection system configured to provide a digitally enhanced representation of a light color transition to the controller.

### BRIEF DESCRIPTION OF THE DRAWINGS

FIG. 1 is a schematic depiction of an exemplary embodiment of a display device.

FIG. 2 is a block diagram of an exemplary embodiment of a display device with a first exemplary embodiment of a color wheel synchronization system.

FIG. 3 is a block diagram of an exemplary embodiment of a display device with a second exemplary embodiment of a color wheel synchronization system.

FIG. 4 is a graphical representation of an exemplary output signal from a digital signal processor of the embodiment of FIG. 3 as a function of time.

FIG. 5 is an exemplary embodiment of a method of synchronizing a light modulator and an image-producing element in a display device.

### DETAILED DESCRIPTION OF THE DEPICTED EMBODIMENTS

One exemplary embodiment of a display device according to the present disclosure is illustrated generally at 10 in FIG. 1 as a projection display device configured to project an image onto a display screen 12. Display device 10 may be either a front projection device or a rear projection device, and may utilize any suitable image producing element to form an image for projection. Examples of suitable image-producing elements include, but are not limited to, liquid crystal displays (LCD), liquid crystal on silicon (LCOS) devices, and digital micromirror devices (DMD). Display device 10 may be a portable display device, permanently installed display device, networked display device, and/or any other suitable display device that utilizes color modulation of a beam of white light to display color images.

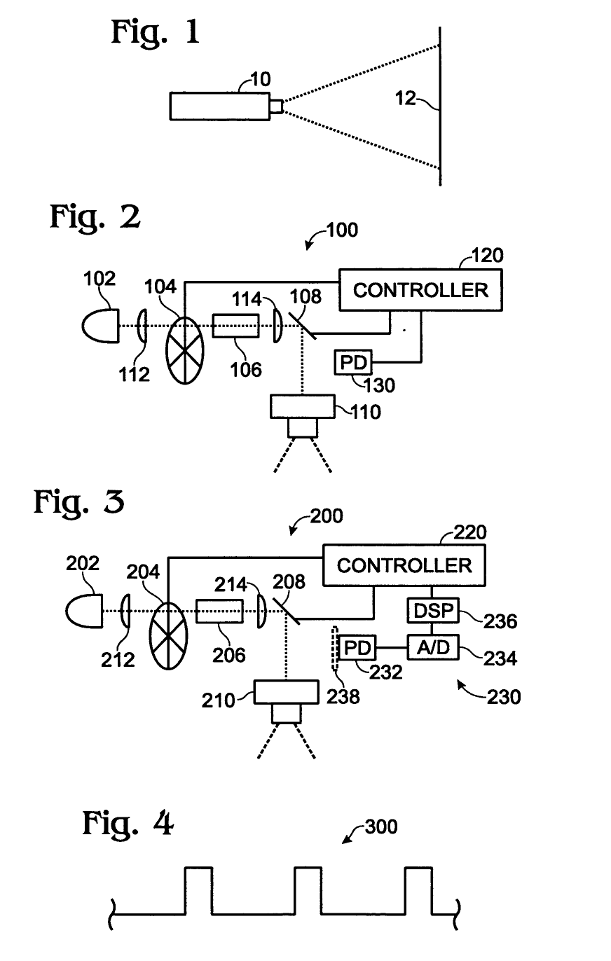

FIG. 2 shows an exemplary block diagram of exemplary internal components 100 of display device 10. Display device 10 includes a lamp 102 for producing white light, and a color wheel 104 for modulating the white light from lamp 102 to produce sequential segments of red, green, and blue (and, in some embodiments, white) light by rotating a series of red, green, and blue color (and, in some embodiments, clear) filters in front of the beam of light from lamp 102. Display device 10 also includes an integrator 106 for conditioning and reshaping the beam of light, an image producing element 108 (for example, a DMD, LCD, or LCOS device) for producing an image from the beam of light, and projection optics 110 for projecting the image produced by image producing element 108. Display device 10 also typically includes other optical components such as lenses 112 and 114, and a controller 120 for receiving an input of image data from an image source, processing the image data, and/or controlling the display of the image data. It will be appreciated that the above-described components are merely exemplary, and that display device 10 may have any suitable subset of these components, and/or any other suitable components not described above. Furthermore, it will be appreciated that color wheel 104 may include additional/other color filters than red, blue and green color filters, and also may include any other suitable optical filters.

As described above, display device 10 displays a color image by sequentially displaying red, green and blue images at a sufficiently high rate that the resulting sequence of separate red, green, and blue images appears to be a single color image. Precise and accurate synchronization of the rotation of color wheel 104 and image producing element 108 such that light of a selected color is incident on image producing element 108 only when image producing element 108 is configured to produce the image of that color allows the production of a high-quality color image. On the other hand, where color wheel 104 and image producing element 108 are not accurately synchronized, mistakes in the appearance and/or color of the image may result.

To synchronize a color wheel with a controller, a photodetector may be positioned in a location optically downstream of the color wheel along the optical path of display device 10 (i.e. such that the color wheel is positioned between the photodetector and the light source), as shown in FIG. 2. The output from the photodetector may be provided to the controller, which may be configured to detect a color transition from the output of the photodetector. Such a photodetector may be positioned in any suitable location in display device 10. In FIG. 2, photodetector 130 is shown positioned next to image producing element 108, and is configured to detect stray light that is beyond the perimeter of image producing element 108. Alternately, photodetector 130 may be positioned in any other suitable location where stray light from downstream of the color wheel has a sufficient intensity to cause a suitable response in the photodetector. For example, U.S. Pat. No. 5,967,636 to Stark et al., the disclosure of which is hereby incorporated by reference, discloses a system in which a photodetector is configured to detect light scattered by the image producing element. While photodetector 130 is shown as being connected directly to controller 120, it will be appreciated that various circuitry, such as amplifiers, filters, etc. may be provided between photodetector 130 and controller 120.

Photodetector 130 may be configured to detect a change in the light beam from lamp 102 caused by color wheel 104 in any suitable manner. For example, in some embodiments, a small black (or otherwise opaque) marking may be provided at a selected border between colors on the color wheel. Photodetector 130 detects the reduction in light intensity as the black marking passes in front of the beam of light. Knowledge of which colors border the black marking and the rotational speed of the color wheel allows both the timing of the transition between colors and the order of the color changes to be determined by controller 120 when the black mark is detected. Controller 120 may then synchronize color wheel 104 and image producing element 108 appropriately.

However, it may be difficult to place such a marking in a consistent location during manufacturing. Such problems may be overcome by calibrating the color wheel in each display device unit during manufacturing. However, this may lead to increased manufacturing time and costs.

Alternatively, in other embodiments, a color filter may be used in front of photodetector 130 to allow a selected color boundary to be detected. This may offers the advantage that the black marking may be omitted, and that calibration during manufacturing may be omitted. However, display systems with high-quality optics may have little stray light within the system. In some display systems, the amount of stray light may be so low that the detection of a boundary between colors becomes difficult for controller 120 to distinguish the transition above a noise level of the photodetector.

FIG. 3 shows a block diagram of an alternate embodiment of internal components 200 of display device 10 having a color transition detection system configured to detect color transitions from stray light of very low intensity. Referring to FIG. 3, display system 200 includes a lamp 202, a color wheel 204, an integrator 206, an image producing element 208, projection optics 210, and other optical components such as lenses 212 and 214. Display system 200 also includes a controller 220.

The color transition detection enhancement system of display system 200 is depicted at 230. Color transition detection enhancement system 230 includes a photodetector 232, an analog-to-digital converter 234 connected to the output of photodetector 232, and a digital signal processor 236 connected to the output of the analog-to-digital (A/D) converter 234. Analog-to-digital converter 234 converts the output from photodetector 232 to digital values, which may then be processed by digital signal processor 236.

Digital signal processor 236 is configured to process the signals from A/D converter 234 in such a manner as to enhance the signals generated by photodetector 232 at color transitions so that the signals are easily distinguished from noise. For example, in one embodiment, a color filter 238 is placed in front of photodetector 232 that causes an increase or decrease in the intensity of light that reaches photodetector 232 as the light beam changes color. Then, the output from photodetector 232 is processed by digital signal processor 236 with specific programming configured to reduce noise and/or enhance the amplitude of the signal caused by a detected transition in light color. The enhanced signal is then output by digital signal processor 236 to controller 220 for color wheel/image producing element synchronization. Alternatively or additionally, color wheel 204 may include one or more opaque markings that cause a reduction in the intensity of light reaching photodetector 232. The change in the photodetector output signal caused by this reduction in light intensity may then be enhanced by digital signal processor 236 for output to controller 220. Furthermore, in yet other embodiments, digital signal processor 236 may be a microprocessor or a field-programmable gate array (FPGA) configured to receive inputs from controller 220, and to synchronize the color wheel accordingly.

FIG. 4 shows an exemplary output signal 300 from digital signal processor 236 as a function of time. Signal 300 as depicted includes a periodic series of output pulses. The output pulses have distinct boundaries and are well above a baseline noise level. The output pulses may be provided to controller 230 for controller 230 to synchronize image producing element 208 to color wheel 204. The output pulses are depicted as a series of square waves, but it will be appreciated that the output from digital signal processor 236 may take any other suitable form. The output signal shown in FIG. 4 may be produced in real-time, or a predetermined time delay may be added prior to the generation of a synchronization pulse.

The use of color transition detection enhancement system 230 may offer several advantages over the system depicted in FIG. 2, as well as other systems and methods for color wheel/image synchronization. For example, controller 220 does not have to rely on the absolute value of the signal from digital signal processor 236. Therefore, the output from digital signal processor 236 may be insensitive to varying light intensities caused by, for example, lamp startup and lamp end-of-life situations. Furthermore, the controller may be configured to synchronize image producing element 208 to color wheel 204 in such a manner that the synchronization will function without calibration or additional programming even when color wheels having different filter sizes are used. This may be helpful when using different color wheels on a single display system platform (for example, for development use, office/home cinema use, etc.), or even color modulators other than color wheels. Furthermore, the use of digital signal processor may be less sensitive to different colors with closely matched digitized values, and may be implemented in a smaller physical space compared to analog solutions.

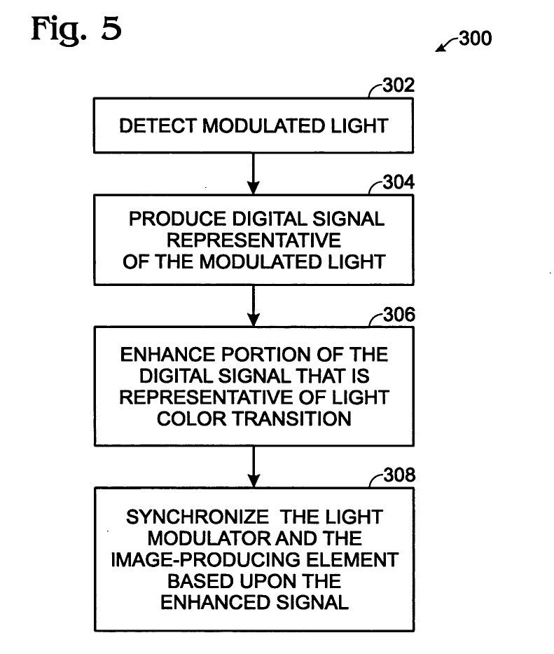

FIG. 5 shows an exemplary embodiment of a method 300 of synchronizing a light modulator and an image-producing element in a display device. Method 300 includes detecting modulated light at 302, producing a digital signal representative of the modulated light at 304, processing the digital signal to enhance a portion of the digital signal that is representative of a light color transition at 306, and synchronizing the light modulator and the image-producing element based upon the enhanced signal at 308. The modulated light may be detected in any suitable manner, including but not limited to the use of a photodetector such as a photodiode, a phototransistor, a photomultiplier tube, etc. Likewise, a digital signal representative of the light color transition may be produced in any suitable manner. For example, in one embodiment, the output of the photodetector may be provided to an analog-to-digital converter to form a digital signal from the analog output. Alternatively, a photodetector that is configured to produce a digital output may be utilized.

The digital signal representing the light color transition may be enhanced in any suitable manner. For example, in display systems having extremely low stray light conditions, the digital signal representing the light color transition may be enhanced by increasing the amplitude of the signal representing the transition or reducing a level of noise in the signal. Furthermore, the signal representing the transition may be delayed before being provided to the controller (wherein the controller may be configured to take this delay into account in synchronizing the image-producing element to the color wheel), the shape of the signal may be modified (for example, a pulse width narrowed or widened, or a peak shape changed), etc. The use of digital signal processing to enhance the signal from the photodetector at a color transition may allow any suitable modification or enhancement to be made to the signal from the photodetector to assist the controller with synchronizing the color modulator and image producing element.

It will be appreciated that the display system embodiments disclosed herein are exemplary in nature, and that these specific embodiments are not to be considered in a limiting sense, because numerous variations are possible. The subject matter of the present disclosure includes all novel and non-obvious combinations and subcombinations of the various display systems, color transition enhancing systems, and other features, functions and/or properties disclosed herein. The following claims particularly point out certain combinations and subcombinations regarded as novel and nonobvious. These claims may refer to “an” element or “a first” element or the equivalent thereof. Such claims should be understood to include incorporation of one or more such elements, neither requiring nor excluding two or more such elements. Other combinations and subcombinations of the display systems, color transition enhancing systems, and/or other features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

What is claimed is:

1. A display system, comprising:
a light source;
an image-producing element;
a light color modulator disposed optically between the light source and the image-producing element;
a controller configured to synchronize the image-producing element and the light color modulator; and
a color transition detection system configured to provide a digitally enhanced representation of a light color transition to the controller.

2. The display system of claim 1, wherein the light color modulator comprises a color wheel.

3. The display system of claim 1, wherein the color transition detection system comprises a photodetector and an analog-to-digital converter configured to receive an analog signal from the photodetector.

4. The display system of claim 3, further comprising a color filter disposed optically in front of the photodetector, wherein the color filter is configured to filter at least one color of light output by the light color modulator.

5. The display system of claim 3, wherein the photodetector is positioned to detect stray light within the projection device.

6. The display system of claim 1, wherein the color transition detection system includes a digital signal processor configured to enhance an electrical signal representing a change in modulated light color.

7. The display system of claim 6, wherein the digital signal processor is configured to increase an amplitude of the electrical signal representing the change in modulated light color.

8. The display system of claim 6, wherein the digital signal processor is configured to reduce a noise level of the electrical signal representing the change in modulated light color.

9. A display system, comprising:
a light source;
an image-producing element;
a light color modulator disposed optically between the light source and the image-producing element;
a controller configured to synchronize the image-producing element and the light modulator;
a photodetector positioned to detect light optically downstream of the light color modulator;
an analog-to-digital converter configured to receive a signal from the photodetector; and
a digital signal processor configured to receive a signal from the analog-to-digital converter, to enhance a portion of the signal from the analog-to-digital converter representing a color transition to form an enhanced signal, and to output the enhanced signal to the controller.

10. The display system of claim 9, wherein the light color modulator comprises a color wheel.

11. The display system of claim 9, wherein the photodetector is positioned to detect stray light.

12. The display system of claim 9, further comprising a color filter disposed optically in front of the photodetector.

13. The display system of claim 9, wherein the digital signal processor is configured to increase an amplitude of the electrical signal representing the color transition.

14. The display system of claim 9, wherein the digital signal processor is configured to reduce a noise level of the signal representing the color transition.

15. In a display device, a method of synchronizing a light modulator and an image-producing element, comprising:
detecting modulated light;
producing a digital signal representative of the modulated light;
processing the digital signal to enhance a portion of the digital signal that is representative of a light color transition, thereby forming an enhanced signal; and
synchronizing the light modulator and the image-producing element based upon the enhanced signal.

16. The method of claim 15, wherein detecting modulated light comprises detecting the modulated light with a photodetector.

17. The method of claim 15, wherein detecting modulated light comprises detecting stray light within the display device.

18. The method of claim 15, wherein producing the digital signal comprises converting an analog signal to a digital signal with an analog-to-digital converter.

19. The method of claim 15, wherein processing the digital signal comprises increasing an amplitude of the portion of the digital signal that is representative of the light color transition.

20. The method of claim 15, wherein processing the digital signal comprises outputting a square wave representative of the light color transition.
